(19) United States
(12) Patent Application Publication
(10) Pub. No.: US 2005/0042265 A1
(43) Pub. Date: Feb. 24, 2005

(54) Composition for cutaneous repair and cicatrization comprising exclusively a true physical hydrogel of chitosan

(75) Inventors: Francois Guillot, Villars-Tiercelin (CH); Alain Domard, Lyon (FR)

Correspondence Address: IP DEPARTMENT OF PIPER RUDNICK LLP, ONE LIBERTY PLACE, SUITE 4900, 1650 MARKET ST, PHILADELPHIA, PA, 19103, US

(73) Assignee: EUROPLAK, a corporation of France, La Garde (FR)

(21) Appl. No.: 10/915,621

(22) Filed: Aug. 10, 2004

Related U.S. Application Data
(63) Continuation of application No. PCT/EP03/01424, filed on Feb. 13, 2003.

(30) Foreign Application Priority Data
Feb. 15, 2002 (FR) 02/01955

(51) Int. Cl. A61K 9/14; A61L 15/00

(52) U.S. Cl. 424/445; 424/488; 514/55

(57) ABSTRACT

A composition for repairing and cicatrization of cutaneous lesions of chronic or acute wounds including a hydrogel of chitosan, the degree of acetylation of which is no greater than about 40%.

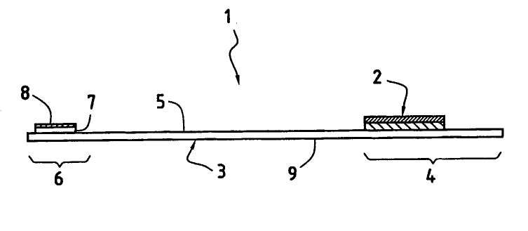

### RELATED APPLICATION

This is a continuation of International Application No. PCT/EP03/01424, with an international filing date of Feb. 13, 2003 (WO 03/068281, published Aug. 21, 2003), which is based on French Patent Application No. 02/01955, filed Feb. 15, 2002.

### FIELD OF THE INVENTION

This invention pertains to a composition and also to a dressing intended for the repair and cicatrization of lesions of the chronic cutaneous wound or acute wound type. The invention pertains more particularly to a composition or a dressing comprising a true physical hydrogel of chitosan.

### BACKGROUND

Lesions of the chronic cutaneous wound and acute wound type are deep lesions that involve both the epidermis and the dermis. Categorized among this type of lesions are decubitus ulcers, venous or arterial ulcers and traumatic wounds as well as burns and acute radiodermatitis. Other dermatological disorders generate cutaneous wounds such as necrotic angiodermatitis, epidermolysis bullosa and grafts.

One applies on such lesions dressings the primary function of which is to protect them from the external environment, but which can also have the secondary function of enhancing the cicatrization process.

Chitin is the most widely distributed biopolymer in nature along with cellulose. Chitin can be extracted from the skeletons of certain animals, e.g., the exoskeletons of certain crustaceans such as lobsters, crabs and shrimp as well as the endoskeleton of squid. Chitin and chitosan comprise two of the same monomer units: N-acetyl-D-glucosamine and D-glucosamine. Chitin contains a high proportion of N-acetyl-D-glucosamine monomer units: greater than 60%. In contrast, chitosan contains a proportion of N-acetyl-D-glucosamine monomer units lower than 60%. Chitosan is notably obtained by total or partial deacetylation of chitin.

Chitin and chitosan have bioactivity properties such as activation of the cicatrization process, biocompatibility, biodegradability and bioresorbability. These properties have resulted in an extensive use of these polymers in the medical field. With regard to the cicatrization of chronic wounds, there has already been proposed in FR 2,736,835 a dressing constituted of an acetylated gel of chitin or chitosan with a degree of acetylation of at least 60%, which corresponds to a chitin gel in accordance with the definition of chitin indicated above. A physical gel is a three-dimensional network of polymer chains linked to each other by reversible (noncovalent) junctions. This definition differs from the definition which incorrectly refers to a very viscous solution as a gel. A hydrogel is a mixture containing a polymer and water at greater than 90%. Thus, in FR 2,736,835, the dressing is a three-dimensional network constituted of a mixture of water and chitin, with the structure of the network being a function of the operating conditions employed. When the dressing is applied to the lesion, there is produced a cellular development which penetrates into the gel, more specifically into the sites left free by the three-dimensional chitin network. Moreover, in the parts of the dressing in contact with the lesion, there is produced a progressive degradation of the three-dimensional network by hydrolysis of the chitin due to the presence of hydrolytic systems such as lysozyme. The objective is to have a progressive consumption of the dressing via biodegradation and bioresorption of the three-dimensional network accompanying the cellular development which should lead to the reconstruction of the dermis and reformation of the epidermis until complete epithelialization. However, the result is difficult to attain under many circumstances.

It would therefore be advantageous to provide a composition as well as a dressing comprising the composition which enables not only protection of a lesion from the external environment, but also cellular reconstruction of the dermis and epidermis until attaining complete cicatrization under optimal conditions.

### SUMMARY OF THE INVENTION

This invention relates to a composition for repairing and cicatrization of cutaneous lesions of chronic or acute wounds including a hydrogel of chitosan, the degree of acetylation of which is no greater than about 40%.

### BRIEF DESCRIPTION OF THE DRAWING

The drawing is a sectional view of one embodiment of the invention selected for illustration.

### DETAILED DESCRIPTION

It will be appreciated that the following description is intended to refer to specific embodiments of the invention selected for illustration in the drawings and is not intended to define or limit the invention, other than in the appended claims.

The invention provides a composition for the repair and cicatrization of cutaneous lesions of the chronic or acute wound type comprising a hydrogel of deacetylated chitosan, the degree of acetylation of which is no higher than about 40%.

The higher the proportion of N-acetyl-D-glucosamine monomer units, the greater the degree of biodegradability and bioresorbability of the chitosan. Thus, in contrast to FR 2,736,835, the composition and the dressing of this invention have the properties of very slow biodegradability and bioresorption.

The degree of acetylation of the chitosan hydrogel is preferably on the order of or less than about 30%. In fact, it has surprisingly been found that there is an abrupt drop in the activity of hydrolytic systems when the degree of acetylation of the chitosan reaches a rather low threshold. It is known that the hydrolytic systems of the lysozyme or N-acetyl-β-d-glucosaminidase type recognize the natural structure of chitin with its monomer units primarily of N-acetyl-D-glucosamine. In contrast, the hydrolytic systems do not recognize a biopolymer comprising exclusively D-glucosamine monomer units. Thus, the greater the degree of deacetylation of the chitosan, the less it is biodegradable and bioresorbable.

In a preferred aspect, the degree of acetylation of the chitosan is between about 2 and about 6%.

The cellular development does not penetrate into the hydrogel according to the invention since the highly deacetylated three-dimensional chitosan network exhibits steric hindrance which prevents the cells from penetrating it. In fact, the size of the surface pores of this type of hydrogel is between about 0.1 and about 5 μm, which is much smaller than the size of the cells.

To the contrary, we found that this cellular development takes place at the interface between the lesion and the hydrogel and that cellular development takes place under optimal conditions. The interface is particularly complex and evolves over the duration of the cicatrization. However, we believe that this interface is constituted of numerous elements, notably by blood products such as fibrin or fibrinogen, von Willebrand factor as well as products released by the activated platelets such as fibronectin, growth factors, PDGF, bFGF, TGF_{α} and TGF_{β}. This interface can thus play a significant role during the inflammatory phase by trapping the polynuclear neutrophils, the monocytes and the factors released by the circulating cells such as TNF_{α}, TGF_{β} and VEGF. Moreover, we believe that this interface still plays an important role during the phase of formation of granulation tissue comprising angiogenesis since similar growth factors are involved (EGF, TNF_{α}, TGF_{β} and PDGF). Finally, we believe that this interface continues its role of cicatrization manager during the final stage corresponding to epithelialization, thereby assisting migration of epithelial cells by binding the growth factors of the EGF family.

We also believe that during this cellular development, a synthesis of specific antibodies of highly deacetylated chitosan takes place which clearly has a beneficial effect on cicatrization. This regulation of the inflammatory phase in the cicatrization process results in a cicatrix that has a very attractive appearance due to the fact that there is no anarchic development of fibroblasts which can lead to keloidal or hypertrophic cicatrices.

In contrast, when the cellular development takes place by penetration and colonization of a three-dimensional network, the orientation and vascularization of the neotissue formed is disturbed which results in defects at the esthetic level.

It is known from EP 0,296,078 to employ in the field of biomaterials chitosan with a degree of acetylation comprised between 10 and 40%. However, the chitosan is combined with collagen and a glycosaminoglycan. This combination leads to the formation of an ionic network with a high degree of cohesion since the three components of the combination have ionizable groups that interact with each other. For example, in the case of using chondroitins 4 and 6 sulfate as glycosaminoglycans, the carboxyl and amine groups of the collagen bind respectively to the amine functional groups of the chitosan and the sulfate and carboxyl groups of the chondroitins 4 and 6 sulfate. Moreover, the acid and sulfate groups of the chondroitins 4 and 6 sulfate can bind ionically to the amine functions of the chitosan.

The biomaterial of EP 0,296,078 does not correspond to a hydrogel, but rather to a lyophilized product of the sponge type. It is entirely biodegradable particularly by collagenase even if the enzymatic degradation rate is reduced by up to 50% because of the presence of chitosan and glycosaminoglycans in the ionic network.

According to this invention, the composition for the repair and cicatrization of cutaneous lesions is maintained constantly in contact with the lesion. The usual practice of changing the dressing should not be performed because that can result in the loss of all of the cellular products in the broadest sense that participate actively in the cicatrization. In fact, since the cellular development takes place at the interface between the lesion and the composition of the invention, removal of the composition can create the risk of destroying locally the beneficial action provided by the highly deacetylated chitosan hydrogel.

The dressing of the invention is preferably in the form of a sheet with a reduced thickness of about 1 to about 10 mm. The sheet may be transparent or at least translucent which allows visual examination of the evolution of the wound at least at the initiation of treatment before certain constituents of the exudates could possibly hinder visual examination.

It has been noted that as long as the epithelialization is not finished, the sheet forming the dressing of the invention adheres lightly to the wound. However, this adherence disappears when the epithelialization is finished which makes possible a particularly easy removal of the dressing at the end of treatment.

Moreover, the cellular reconstruction induced by the highly deacetylated chitosan hydrogel yields a neotissue that is oriented both in terms of collagen fibers and vascularization. This neotissue has a remarkable appearance without hypergranulation. We also observed that, when employing the composition of the invention, cicatrization does not take place solely along the edges of the lesion and that, furthermore, formation of a scab is substantially prevented. We believe that this remarkable cellular reconstruction is due to the trapping of growth factors by the highly deacetylated chitosan hydrogel. In the case of superficial and deep second degree burns in particular, application of the composition of the invention facilitates cellular development from the reserves of stem cells located at the base of hair follicles.

Application of the composition of the invention in the form of a sheet creates surface contact. It is particularly suitable when the lesion is a shallow cavity and largely accessible. It is desirable that the sheet exhibit good mechanical strength so that it is not deformed or degraded during handling/application.

The sheet is formed exclusively by a hydrogel of chitosan having a degree of acetylation of no more than about 40%. It is preferable that its molar mass be at least about 400,000 g/mol. The mechanical resistance of the gel is notably dependent on this molar mass. At this threshold, the sheet with a thickness of about 1 to about 10 mm has sufficient strength to be handled/applied. The molar mass value may be measured using the light diffusion technique and not by measurement of viscosity. This latter technique is now considered to yield erroneous results.

More specifically, the mean molar mass in weight (Mw) of the polymer may be determined from the light diffusion measurements at 18 different angles using the Dawn DSP-EOS spectrometer (Wyatt). The mean molar mass is then reduced using a Zimm diagram by means of values calculated with the Rayleigh-Deybe equation. This method uses knowledge of the refraction index increment of the polymer (dn/dc) in the solvent used for the preceding experiment. This parameter is measured using an interferometer operating at the same wavelength as the light diffusion device.

Since the repair and cicatrization effect takes place at the interface between the gel and the lesion it may be possible to reduce the thickness of the sheet if only for cost-saving reasons. However, such a thinner sheet may be more fragile and difficult to handle. To overcome this potential drawback, a dressing in the form of a chitosan hydrogel sheet as above of reduced thickness, e.g., from about 1 to about 3 mm, may be supported by a support material preferably based on cellulose, notably made of paper. In this case, the mechanical strength of the dressing is principally provided by the support material which, moreover, prevents deformation of the sheet in the case that stress is applied to the dressing. The fact that the support material is based on cellulose avoids allergenic phenomena. Cellulose is a natural polymer that does not have contraindications from a biological point of view.

The support preferably is of a dimension larger than that of the sheet, preferably of proportions allowing it to be wound around a limb. In this latter case, the support can at the external zones of the sheet be provided with attachment means so that it can be held in place after being wound around a limb. These means can be notably adhesive or self-adhering.

The assembly of the sheet and the support is implemented by impregnation of the support material by the chitosan solution during formation of the gel. This process can involve surface impregnation with partial or total diffusion of the gel on either side of the support.

Since paper is naturally hydrophilic, it absorbs water from the chitosan solution during the previously mentioned impregnation. This tends to concentrate—at least locally—the chitosan hydrogel. This can be avoided by wetting the paper during impregnation or by selecting as a support material an artificial parchment paper or a sheet of a hydrophobic material or a material that has been made hydrophobic. In the case of parchment paper, its pores are blocked and it retains a certain hydrophilic character which enables better control of the hydration of the wound.

This presentation in sheet form—with or without support material—is not restrictive of the invention. The highly deacetylated chitosan hydrogel can be presented in the form of particles of more or less reduced size, e.g., obtained by grinding a preformed sheet.

In one particularly advantageous mode of presentation, these particles are made available in the form of a paste packaged in a suitable container, e.g., a tube or a syringe. To produce such a paste, a chitosan hydrogel (as described above) is ground in water and then the water is partially eliminated by centrifugation until the paste is obtained.

The paste can be introduced into a cavity and remain there until cicatrization. In this case, if the cavity is open to the outside, there is no bioresorption of the hydrogel and the hydrogel is progressively pushed to the outside as the reconstruction of the neotissue progresses.

### EXAMPLE

The description below pertains to a preferred example of preparation of a true physical gel of chitosan that has a degree of acetylation of between 2 and 6% in the form of a sheet that can be used as a dressing for the repair and cicatrization of cutaneous lesions of the chronic or acute wound type. These lesions may be in humans or animals.

The initial chitosan used to produce this hydrogel has a degree of acetylation between 2 and 6% and a viscosity greater than 4000 cps (1% solution in 99° acetic acid at 1%, T=25° C.). This is obtained by controlled deacetylation steps from chitin purified from squid endoskeletons. Each deacetylation step takes place in a caustic bath at an elevated concentration of soda.

After dissolution, a solution of weak viscosity is prepared (0.5% by weight of chitosan in an acid solution). Three filtration steps using membranes of decreasing porosity (1.2, 0.8 and 0.45 μm) under a maximal pressure of 3 bars are applied to the polymer.

The polymer solution filtered in this manner is then precipitated by addition of a concentrated ammonia solution. This precipitate is then washed multiple times to eliminate the excess ammonia. After stabilization of the pH of the washing water, the resultant product is lyophilized thereby producing the chitosan in solid form.

After dissolution of the solid chitosan, preferably in acetic acid medium, 1,2-propanediol is added drop by drop. This is then rapidly degassed under vacuum for a duration of approximately one hour.

The formation of the true physical gel of chitosan according to the invention corresponds to the passage from a solution state (prior situation) to a physical gel or hydrogel state for which the physical cross-linkings between chains are formed from hydrophobic interactions and hydrogenic bonds. The solution becomes a gel and there is thereby produced a chitosan gel having a degree of acetylation between 2 and 6% and a chitosan concentration between 0.2 and 5%.

The size of the gel is dependent on the size of the mold selected. For a favorable gelification at T=45° C., it is preferable to select a mold having a large surface which facilitates partial evaporation of the water and the acetic acid, thereby leading to gelification.

Neutralization of the resultant hydrogel is performed by transfer to a basic medium. This step prevents solubilization of the hydrogel in water at physiological pH or when the hydrogel is in contact with physiological solutions or media. The hydrogel is lastly subjected to successive rinsings to eliminate the di-alcohol and obtain a pH value close to 7.

The hydrogel is preferably sterilized by autoclaving in moist heat (T=121° C., 30 min). It is plunged into a vessel containing water or a selected buffered solution, and this is placed in the autoclave. After autoclaving, the water or buffered solution is carefully poured or aspirated under a sterile hood. The gel can then be packaged in a sterile manner, especially in a sterile blister pack, until its future use.

It is important to emphasize that substantially no chemical cross-linking (formation of covalent bonds) takes place during the formation of the chitosan hydrogel. This gelification is created by a substantially purely physicochemical process. The resultant product is a true physical gel of chitosan.

The single figure shows in section one aspect of the invention that includes a dressing 1 comprising a sheet 2 formed by a chitosan gel having a degree of acetylation of no more than about 40% and a cellulose-based paper support 3 supporting said sheet 2. The sheet 2 has reduced dimensions (for example, 3 cm×3 cm) in relation to the dimensions of the paper 3 (for example 20 cm×5 cm) and is placed in a median manner at an end zone 4 of the paper 3. On the surface 5 supporting the sheet 2, at the other end zone 6 is provided a pressure-sensitive adhesive coating 7 protected by a protective anti-adhesive strip 8. The dressing 1 with the sheet 2 is positioned to cover the lesion on the arm, wraps the arm with the support paper 3 until covering the two end zones 4, 6 after having removed the protective strip 8. The dressing 1 is then held in place around the arm with the adhesive coating 7 adhering on the other surface 9 of the support paper 3.

1. A composition for repairing and cicatrization of cutaneous lesions of chronic or acute wounds comprising a hydrogel of chitosan, the degree of acetylation of which is no greater than about 40%.

2. The composition according to claim 1, wherein the degree of acetylation of the chitosan hydrogel is about 30% or less.

3. The composition according to claim 2, wherein the degree of acetylation of the chitosan hydrogel is between about 2 and about 6%.

4. A dressing comprising a sheet containing the composition according to claim 1, wherein the sheet has a thickness from about 1 to about 10 mm.

5. The dressing according to claim 4, wherein the sheet has a molar mass measured by light diffusion technique of at least about 400,000 g/mol.

6. The dressing according to claim 4, wherein the sheet of chitosan hydrogel is supported by a support material.

7. The dressing according to claim 6, wherein the support material is an artificial parchment paper or a hydrophobic material or a material that has been made hydrophobic.

8. The dressing according to claim 6, wherein the support has a dimension greater than that of the sheet in proportions that allow it to be wound around a limb.

9. The dressing according to claim 8, wherein the support, in zones external to the sheet, has attachment means which hold the support in place after being wound around a limb.

10. A dressing comprising the composition according to claim 1, in the form of small particles.

11. The dressing according to claim 10, wherein the particles are ground particles in a paste.

12. A method of preparing the dressing of claim 11, comprising preparing the composition for repairing and cicatrization of cutaneous lesions of chronic or acute wounds comprising a hydrogel of chitosan, the degree of acetylation of which is no greater than about 40%, by grinding the hydrogel in water and partially eliminating the water to obtain the paste.

13. A composition for repairing and cicatrization of cutaneous lesions of chronic or acute wounds consisting essentially of a hydrogel of chitosan, the degree of acetylation of which is no greater than about 40%.

14. The composition according to claim 13, wherein the degree of acetylation of the chitosan hydrogel is about 30% or less.

15. The composition according to claim 14, wherein the degree of acetylation of the chitosan hydrogel is between about 2 and about 6%.

16. A dressing comprising a sheet containing the composition according to claim 13, wherein the sheet has a thickness from about 1 to about 10 mm.

17. The dressing according to claim 16, wherein the sheet has a molar mass measured by light diffusion technique of at least about 400,000 g/mol.

18. The dressing according to claim 16, wherein the sheet of chitosan hydrogel is supported by a support material.

19. The dressing according to claim 18, wherein the support material is an artificial parchment paper or a hydrophobic material or a material that has been made hydrophobic.

20. The dressing according to claim 18, wherein the support has a dimension greater than that of the sheet in proportions that allow it to be wound around a limb.

21. The dressing according to claim 20, wherein the support, in zones external to the sheet, has attachment means which hold the support in place after being wound around a limb.

22. A dressing comprising the composition according to claim 13, in the form of small particles.

23. The dressing according to claim 22, wherein the particles are ground particles in a paste.

24. A method of preparing the dressing of claim 23, comprising preparing the composition for repairing and cicatrization of cutaneous lesions of chronic or acute wounds comprising a hydrogel of chitosan, the degree of acetylation of which is no greater than about 40%, by grinding the hydrogel in water and partially eliminating the water to obtain the paste.
